(19) United States
(12) Patent Application Publication
(10) Pub. No.: US 2024/0219288 A1
(43) Pub. Date: Jul. 4, 2024

(54) DEVICE AND METHOD FOR OBSERVING FLUORESCENCE OR LUMINESCENCE OF A MOVING PARTICLE

(71) Applicant: COMMISSARIAT A L'ENERGIE ATOMIQUE ET AUX ENERGIES ALTERNATIVES, Paris (FR)

(72) Inventor: Ondrej MANDULA, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 18/397,259

(22) Filed: Dec. 27, 2023

(30) Foreign Application Priority Data
Dec. 27, 2022 (FR) 22 14523

(51) Int. Cl. G01N 15/149; G01N 15/01; G01N 15/10; G01N 15/1433; G01N 15/1434

(52) U.S. Cl. CPC G01N 15/149; G01N 15/01; G01N 15/1433; G01N 15/1434; G01N 2015/1027

(57) ABSTRACT

Method for observing an emission of fluorescence light or luminescence light from a moving particle in a sample, in a spectral emission band, the method comprising:
a) forming a detection image of the sample in a spectral detection band, the spectral detection band being different from the spectral emission band;
b) forming an emission image of the sample in the spectral emission band;
   the method being such that:
steps a) and b) are reiterated;
the method comprising:
c) on the basis of each detection image resulting from step a) of each iteration of steps a) and b), detecting the particle and determining a region of interest around the particle;
d) on the basis of the region of interest resulting from each step c), extracting a region of interest from each emission image of the sample.

### TECHNICAL FIELD

The technical field of the invention relates to the observation of fluorescence or luminescence of a moving particle.

### PRIOR ART

The use of fluorescence imaging is a well-known technique for characterizing particles, especially in the field of biology. However, fluorescence light often has a low intensity. A current practice is to acquire a fluorescence image over a long acquisition time, or to cumulate different fluorescence images. This can provide a fluorescence image whose signal-to-noise ratio is acceptable.

FIG. 1 shows a sample comprising spermatozoa which are mobile. For such particles, the usual practices of increasing the acquisition time or cumulating different images are unsuitable. This is because the particles move along a trajectory that is difficult to predict. And each particle follows a different trajectory from any other particle.

The invention described below enables this problem to be overcome. It enables the fluorescence of moving particles to be observed. The invention is equally applicable to the observation of the luminescence of a moving particle.

### SUMMARY OF THE INVENTION

A first object of the invention is a method for observing an emission of fluorescence light or luminescence light from a moving particle in a sample, the particle emitting the fluorescence or luminescence light in a spectral emission band, the method comprising:
a) illuminating the sample in a spectral detection band, and forming a detection image of the sample in the spectral detection band, the spectral detection band being different from the spectral emission band;
b) forming an emission image of the sample in the spectral emission band;
the detection image and the emission image being obtained on the basis of an acquisition of an image of the sample by an image sensor, in the spectral detection band and in the spectral emission band,
   wherein:
steps a) and b) are reiterated;
the method also comprises the following steps:
c) on the basis of each detection image (I_{d}) resulting from step a) of each iteration of steps a) and b), detecting the particle and determining a region of interest around the particle;
d) on the basis of the region of interest resulting from each step c), extracting a region of interest from each emission image of the sample, the region of interest extracted from each emission image corresponding to the same particle as that detected in each detection image;
e) summing the regions of interest extracted in each step d) so as to form an integrated emission image of the particle, representative of the fluorescence or luminescence of the particle.

In one embodiment:
The particle emits a fluorescence light in the spectral emission band when it is illuminated in a spectral excitation band:
step b) includes illuminating the sample in the spectral excitation band;
the spectral detection band is remote from the spectral excitation band;
the image sensor is coupled to a filter so as to block the spectral excitation band.

Steps a) and b) may be executed simultaneously.

Steps c), d) and e) may be executed during each iteration of steps a) and b), or after the iterations of steps a) and b).

In each iteration, steps a) and b) may be executed successively.

According to one option:
At least two iterations of steps a) and b) are executed successively, according to an iteration of rank n and an iteration of rank n+1, where n is a strictly positive integer;
step d) comprises:
(i) interpolating the positions of the particle in the detection images resulting from steps a) of the iterations of rank n and n+1, so as to estimate an interpolated position of the particle in step b) of the iteration of rank n;
(ii) extracting the region of interest from the emission image formed in the iteration of rank n on the basis of the interpolated position estimated in sub-step (ii).

In one embodiment:
The image sensor is a colour image sensor;
in step a), the detection image of the sample is obtained on the basis of a first spectral component of the image acquired by the image sensor, the first spectral component corresponding to all or part of the spectral detection band;
and/or, in step b), the emission image of the sample is obtained on the basis of a second spectral component of the image acquired by the image sensor, the second spectral component corresponding to all or part of the spectral emission band;
in such a way that the same image acquired by the image sensor may be used to form the detection image and the emission image of the sample.

In one embodiment:
the image sensor comprises a first elementary image sensor and a second elementary image sensor, together with a beam splitter, the beam splitter being configured to send light,
in the spectral detection band, towards the first elementary image sensor;
and in the spectral emission band, towards the second elementary image sensor;
in each step a), the detection image, in the spectral detection band, is acquired by the first elementary image sensor;
in each step b), the emission image, in the spectral emission band, is acquired by the second elementary image sensor.

According to one option, step e) comprises calculating an average of the regions of interest extracted from each emission image of the sample corresponding to the same particle.

According to one option, step e) is executed in each iteration of steps a) and b).

According to one option:
the stop criterion is a predetermined number of steps;
or, step e) being executed in each iteration of steps a) and b), the stop criterion is the obtaining of an integrated emission image in which the signal-to-noise ratio exceeds a predetermined threshold,
or, step e) being executed in each iteration of steps a) and b), the method comprises a display of the integrated emission image, the iterations being stopped by a user.

According to one option, in each step a), the intensity of illumination of the sample in the spectral detection band is adjusted so that the signal-to-noise ratio of the detection image is less than a predetermined value.

A second object of the invention is a device for observing a sample comprising a moving particle, the particle being capable of emitting a fluorescence or luminescence light in a spectral emission band, the device comprising:
a detection light source, configured to illuminate the sample in a spectral detection band, remote from the spectral emission band;
an image sensor configured to acquire an image of the sample, in the spectral detection band and the spectral emission band;
the device being configured to keep the sample facing the image sensor on a sample plane;
an emission filter, placed between the image sensor and the sample plane, the emission filter being configured to transmit light in the spectral detection band and in the spectral emission band;
a processing unit (30), configured to form, on the basis of the image acquired by the image sensor,
a detection image of the sample, in the spectral detection band;
an emission image of the sample, in the spectral emission band;
the device being such that the processing unit is programmed to execute steps a) to e) of a method according to the first object of the invention.

The device may comprise an excitation light source, configured to illuminate the sample in a spectral excitation band, remote from the spectral emission band and the spectral detection band.

According to one option, the detection light source and the excitation light source are configured to be activated simultaneously, or sequentially with a time shift of less than 100 ms or 10 ms.

According to one option, the image sensor is a colour image sensor.

According to one option, the image sensor comprises a first elementary image sensor and a second elementary image sensor, together with a beam splitter, the beam splitter being configured to transmit light,
in the spectral detection band, towards the first elementary image sensor;
and in the spectral emission band, towards the second elementary image sensor;
   The invention will be more readily understood from a perusal of the examples of embodiment set out in the remainder of the description, with reference to the figures listed below.

### FIGURES

FIG. 1 shows a sample comprising spermatozoa.

FIG. 2A shows a first embodiment of a device according to the invention.

FIG. 2B shows a second embodiment of a device according to the invention.

FIG. 3 shows the transmission pass bands of different filters that may be used in the device.

FIG. 4A shows an image of spermatozoa in a spectral detection band.

FIG. 4B shows an image of spermatozoa in a spectral fluorescence band.

FIG. 5A shows a sequence of steps in the processing of detection and fluorescence images of a sample.

FIG. 5B shows another possible sequence of steps in the processing of detection and fluorescence images of a sample.

FIG. 5C shows another possible sequence of steps in the processing of detection and fluorescence images of a sample.

FIG. 6A shows a region of interest corresponding to a spermatozoon, determined on the basis of a detection image.

FIG. 6B shows the region of interest defined in image 6A applied to a fluorescence image.

FIG. 7 shows different regions of interest formed on different detection images (left-hand column) and extracted from different fluorescence images (right-hand column). Each region of interest corresponds to the same spermatozoon. Each line in this figure shows a detection image and a fluorescence image obtained simultaneously.

FIGS. 8A, 8B, 8C and 8D show the formation off an integrated fluorescence image of a spermatozoon.

FIG. 9A shows schematically an embodiment in which the detection and fluorescence images are obtained from images acquired successively by the image sensor.

FIG. 9B shows an estimate of the position of a region of interest corresponding to a spermatozoon at one instant, based on the positions of the spermatozoon determined from detection images acquired before and after said instant. In FIG. 9B, the circled positions correspond to true positions. The positions represented by a cross are positions interpolated on the basis of two true positions preceding and following each interpolated position respectively.

FIGS. 10A, 10B, 10C, 10D and 10E show an example of the application of the invention to the determination of the condition (living or dead) of a spermatozoon.

FIGS. 11A, 11B, 11C, 11D and 11E show an example of the application of the invention to the determination of the condition (living or dead) of a spermatozoon.

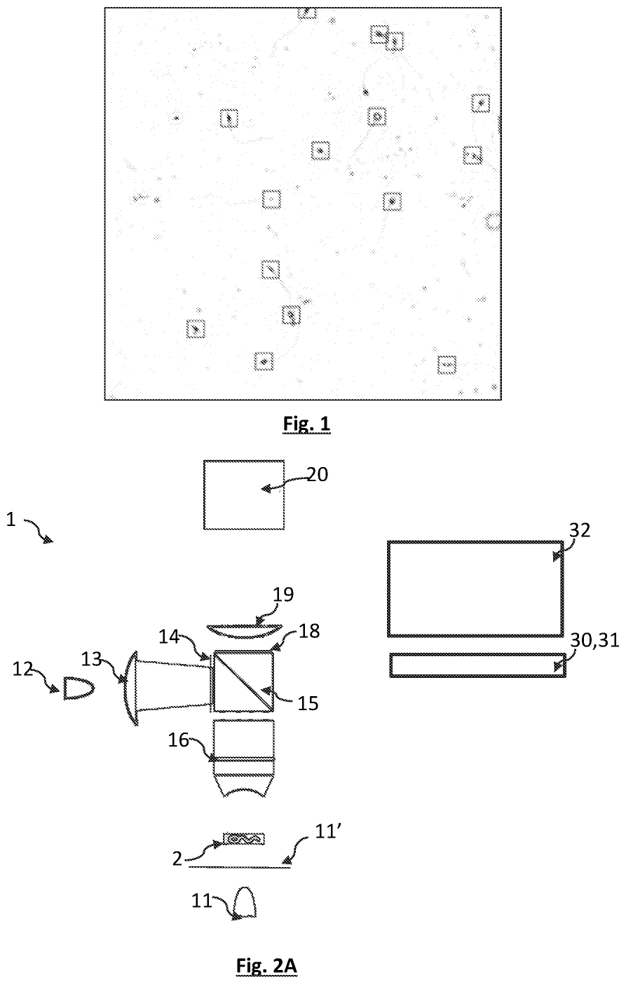

### DISCLOSURE OF PARTICULAR EMBODIMENTS

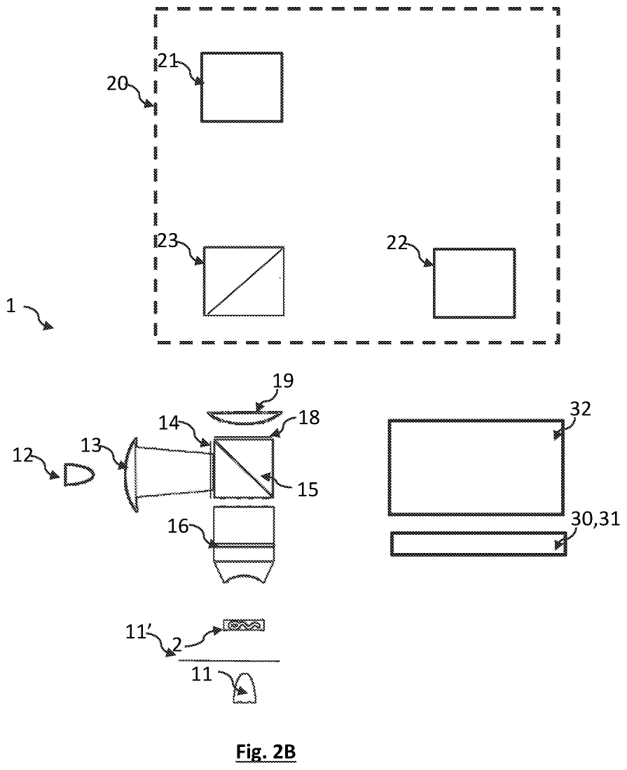

FIG. 2A shows an example of a device 1 for implementing the invention. The device is designed to observe a sample 2 comprising moving particles. In this example, but not exclusively, the particles are biological particles, particularly spermatozoa. The spermatozoa are marked with a fluorophore. The fluorophore may be a viability marker, a sexing marker, an apoptosis marker, or a physiological marker. The fluorophore emits a fluorescence light in a spectral emission band Δλm when it is illuminated in a spectral excitation band Δλe. Spermatozoa may be marked with different fluorophores, each fluorophore having a spectral excitation band Δλe and a fluorescence spectral emission band Δλm. For a single fluorophore, the fluorescence spectral emission band Δλm is remote from the spectral excitation band Δλe, the overlap between the fluorescence spectral emission band Δλm and the spectral excitation band being assumed to be zero.

The device comprises:
a detection light source 11, configured to emit light in a spectral detection band Δλd. The spectral detection band Δλd is different and remote from the spectral excitation band Δλe and from the fluorescence spectral emission band Δλm of the fluorophore marking the spermatozoon;
an excitation light source 12, configured to emit excitation light in the spectral excitation band Δλe of the fluorophore;
an image sensor 20, designed to acquire an image of the sample. The image sensor 20 is a colour image sensor, enabling an image to be formed according to different spectral components. The image sensor comprises pixels forming a matrix of pixels extending along a detection plane.
an objective lens 16, configured to concentrate the excitation light, reflected by the beam splitter cube 15, on to the sample. The objective lens 16 defines an image plane and an object plane. Preferably, the object plane extends in the sample, and the image plane corresponds to the detection plane of the image sensor 20. According to one option, called a defocused imaging option, the detection plane is slightly offset relative to the image plane of the objective lens 16, and/or the object plane is slightly offset relative to the sample. This procedure enables a holographic image to be formed. By using appropriate reconstruction algorithms, a phase image of the sample can then be produced. Defocused imaging is generally used when the particles of the sample are, or are considered to be, transparent. However, for the observation of fluorescence light, it is preferable to opt for the focused configuration, in which the sample extends in the object plane of the objective lens and the image sensor extends along the image plane of the objective lens. The defocused configuration may be used to form the detection images described below.

In the example shown here, the device comprises:
a beam splitter cube 15, comprising a dichroic plate, for directing the excitation light towards the sample 2 through the objective lens 16;
a converging lens 13, positioned facing the second light source 12. The converging lens may be used to shape the excitation light towards the beam splitter cube 15;
a converging lens 19, for focusing the light from the sample towards the image sensor.

The device may comprise filters for adjusting the spectral detection band Δλd and the spectral excitation band Δλe, and for defining the spectral bands that reach the image sensor 20. The device is configured so that the image sensor cannot detect the spectral excitation band.

In this example, a detection filter 11′ is placed between the detection light source 11 and the sample. The detection filter 11 is configured to define the spectral detection band Δλd. The use of the detection filter is optional, particularly when the light source 11 emits light in a well-defined spectral detection band, as is the case, for example, with a light-emitting diode (LED).

The device comprises an excitation filter 14, for delimiting the spectral excitation band Δλe of the light emitted by the excitation light source 12.

The device comprises an emission filter 18, for blocking the (or each) spectral excitation band, so as to make the image sensor insensitive to the latter. The emission filter 18 passes light for the spectral detection band Δλd and for the (or each) fluorescence spectral emission band Δλm. The emission filter is placed between the beam splitter cube 15 and the lens 19.

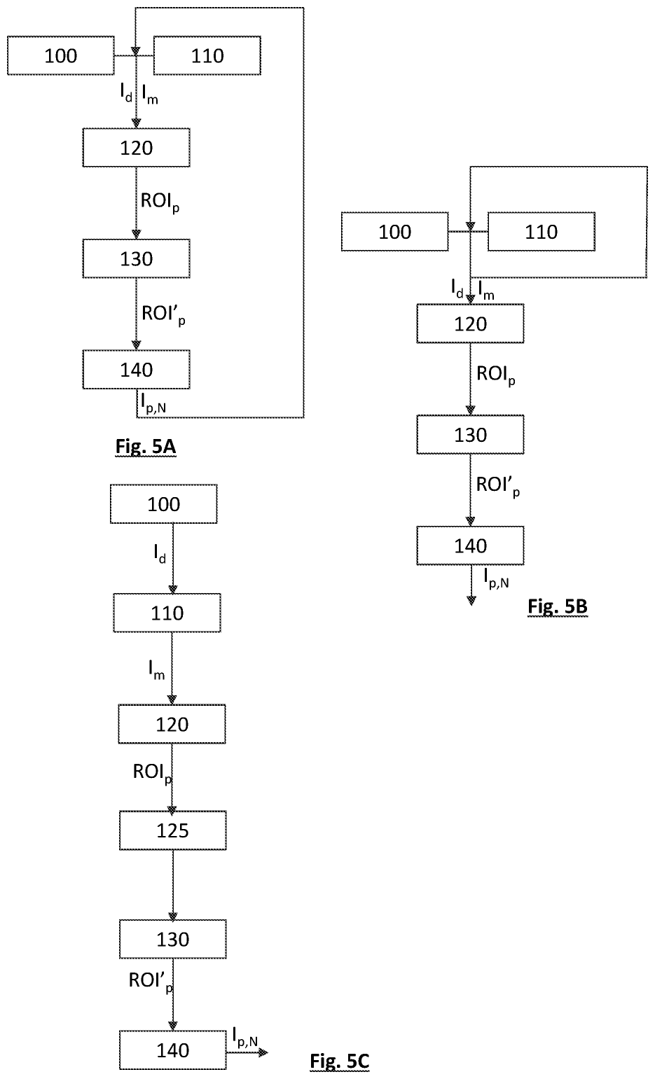

The device comprises a processing unit 30, comprising a microprocessor programmed to execute image processing operations described below with reference to FIGS. 5A to 5C. The image processing operations are programmed and stored in a memory 31 to which the processing unit is connected. The processing unit may be connected to a screen 32.

In the device described with reference to FIG. 2A, the detection light source 11 is separate from the excitation light source 12. The intensity of emission of the detection light source 11 may be adjusted to prevent any of the intensity of the detection light source being detected in the spectral emission band Δλm by cross-talk. The adjustment consists in obtaining a sufficient signal-to-noise ratio to correctly detect the position of each spermatozoon in the detection images. This is a matter of “minimizing” the intensity of the detection light source so as to obtain a signal-to-noise ratio above an acceptable threshold.

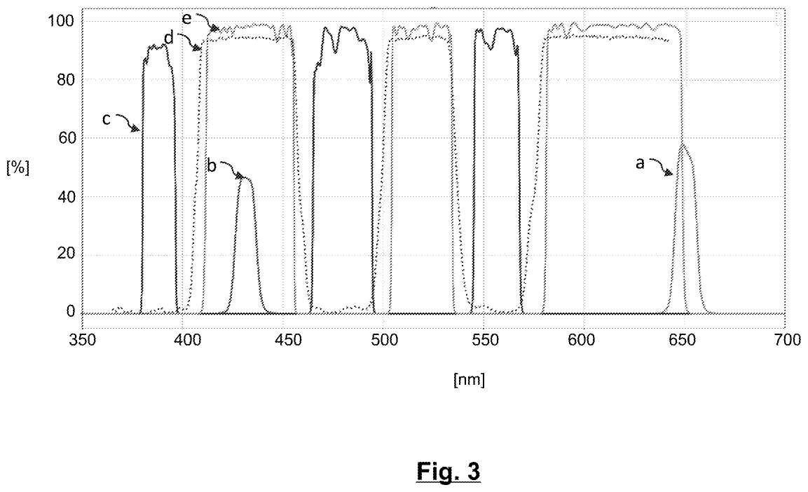

FIG. 3 shows different filter pass bands that may be used. Curve a shows the spectral band of a detection filter 11′ (reference, Thorlabs 650-10) defining a pass band centred on 650 nm. Curve b shows the spectral band of another detection filter 11′ (reference, Thorlabs 430-10) defining a pass band centred on 430 nm. Curve c shows the spectral band of an excitation filter 14. Curve d shows the spectral transmission band of the dichroic mirror included in the cube 15. Curve e shows the spectral transmission band of the excitation filter 18.

Curves c, d and e were obtained with a Semrock DA/FI/TR/3X-A filter set. Curves c, d and e show the possibility of simultaneously defining a plurality of spectral excitation bands Δλe (see the pass bands of curve c) and a plurality of fluorescence spectral emission bands Δλm (see the pass bands of curves d and e, which block the spectral excitation bands). This makes it possible to deal simultaneously with a plurality of fluorophores having different fluorescence spectral bands Δλm from one another. This also makes it possible to use one of the pass bands defined by the emission filter to transmit the spectral detection band Δλd.

In the embodiment shown in FIG. 2A, the image sensor 20 is a colour image sensor. Thus the same image sensor may be used for acquiring the detection image I_{d}, in the spectral detection band Δλd, and a fluorescence emission image Iₘ in the fluorescence spectral emission band Δλm.

The term “detection image I_{d}” is taken to mean an image for detecting the position of the spermatozoon in the sample. The detection image I_{d} corresponds to a spectral component of the image acquired by the image sensor, in the spectral detection band Δλd.

The fluorescence emission image Iₘ corresponds to a spectral component of the image acquired by the image sensor, in the fluorescence spectral emission band Δλm. If it is desired to deal simultaneously with a plurality of fluorescence emission images Iₘ, each fluorescence emission image corresponds to a spectral component of the image acquired by the image sensor, in a fluorescence spectral emission band Δλm.

FIG. 2B shows another example of a device according to the invention, in which the image sensor 20 is formed by a first elementary image sensor 21 and a second elementary image sensor 22, separated from one another. In this configuration, the image sensor comprises a beam splitter 23 configured for:
directing the light emitted by the sample, in the spectral detection band Δλd, towards the first elementary image sensor 21;
directing the light emitted by the sample, in the fluorescence spectral emission band Δλm, towards the second elementary image sensor 22.

The detection image I_{d} corresponds to the image acquired by the first elementary image sensor 21. The fluorescence emission image I_{d} corresponds to the image acquired by the second elementary image sensor 22.

The inventor has implemented a configuration as described with reference to FIG. 2A, the colour image sensor being an RGB IDS UI-3160CP-C-HQ sensor. The objective lens 16 was an objective lens with a magnification of ×20—digital aperture 0.4—reference Olympus Plan Achromat 20×/0.4NA. The filter set described with reference to FIG. 3 was used. The sample 2 comprised spermatozoa.

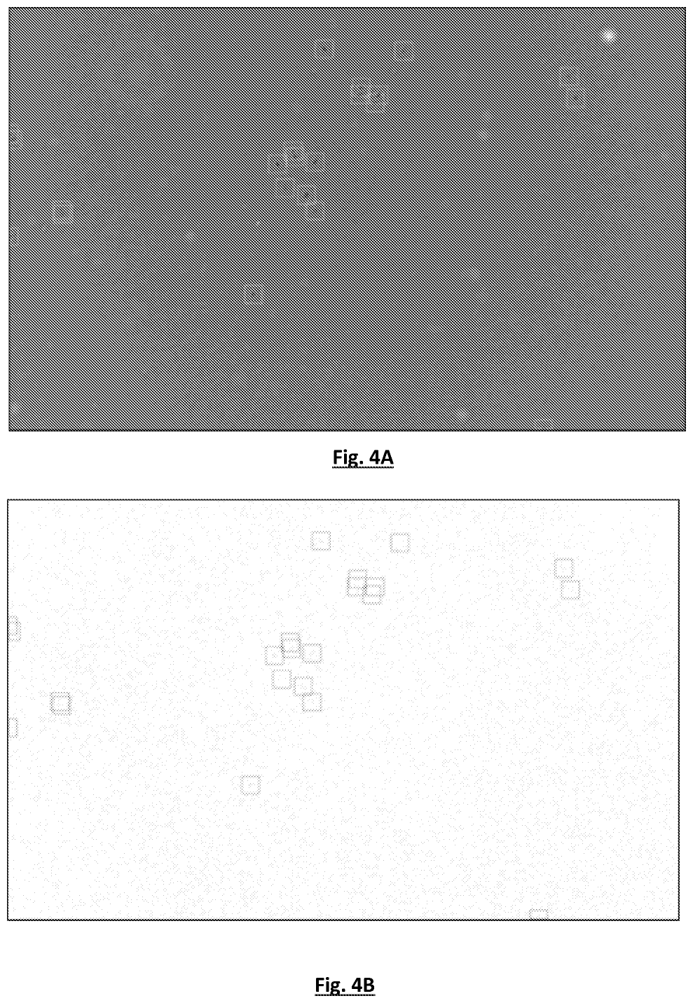

In FIGS. 4A, 4B, 6A, 6B, 7, 8A, 8B and 8C, the images are shown inverted, with the dark grey levels corresponding to high levels of luminous intensity.

In a first series of tests, the spectral detection band Δλd was in the red spectral band, centred on 650 nm (Thorlabs FB650-10 filter). The spermatozoa were marked with a Hoechst fluorescent marker. The excitation light source 12 was an LED emitting at 375 nm. To allow for the movement of the spermatozoa, the images were acquired with a short exposure time, namely 10 ms.

FIG. 4A shows an example of a detection image I_{d} formed, on the basis of the image acquired by the image sensor, in a spectral component corresponding to the spectral detection band Δλd centred on 650 nm. FIG. 4B shows the fluorescence image Iₘ formed, on the basis of the image acquired by the image sensor, in a spectral component corresponding to the fluorescence spectral emission band Δλm.

The detection image I_{d} and the fluorescence image Iₘ correspond to the components of the same image acquired by the image sensor in the spectral detection band Δλd and the fluorescence spectral emission band Δλm, respectively.

The position of each spermatozoon in the detection image was detected (see FIG. 4A). A region of interest ROI was defined around each spermatozoon, as indicated by light-coloured frames in FIG. 4A.

It may be observed that the fluorescence signal is particularly weak: see FIG. 4B. A single fluorescence emission image is not sufficient to form an image of acceptable quality. This is the result of both the short exposure time and the low fluorescence yield. FIG. 4B shows the regions of interest ROI′ extracted, in the fluorescence emission image, on the basis of the positions of the spermatozoa found in the detection image (FIG. 4A).

In order to obtain a more usable fluorescence signal from each spermatozoon, the fluorescence signal emitted by each spermatozoon must be integrated over a longer period. It is therefore necessary to overcome the difficulty arising from the movement of the spermatozoa. This is the aim of steps 100 to 140 shown schematically in FIGS. 5A to 5C.

Step 100: illuminating the sample in the spectral detection band, using the detection light source 11, and acquiring, using the image sensor, a detection image of the sample in the spectral detection band Δλd. In this example, the image sensor is a colour image sensor, the device used being that described with reference to FIG. 2A.

Step 110: illuminating the sample in the spectral excitation band Δλe, using the detection light source 12, and acquiring, using the image sensor, a fluorescence image of the sample in the fluorescence spectral emission band Δλm. This is the same image sensor as that used in step 100.

In this example, steps 100 and 110 are executed simultaneously. The sample is illuminated continuously by the detection light source and by the excitation light source. The same image I is acquired simultaneously in the spectral detection band Δλd and in the fluorescence spectral emission band Δλm. This is an advantageous embodiment, since it requires only one colour image sensor.

Step 120: forming a detection image I_{d} of the sample, on the basis of the image I acquired by the image sensor. The detection image I_{d} corresponds to a first spectral component, in the spectral detection band Δλd, of the image I acquired by the image sensor.

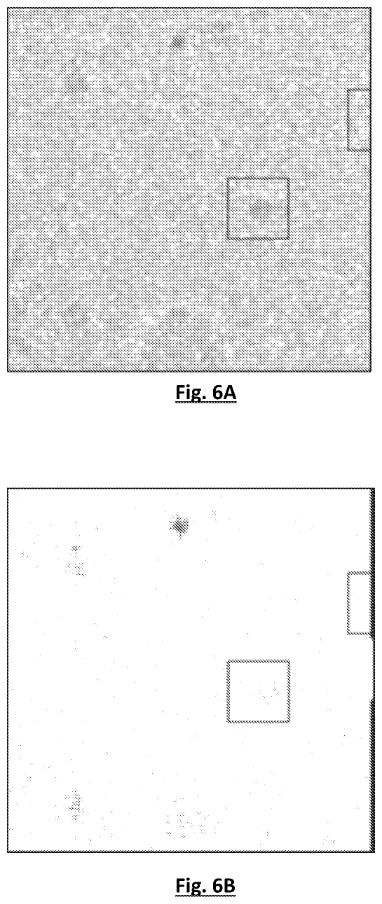

On the basis of the detection image I_{d}, detecting the spermatozoa and determining a region of interest (ROI) around each spermatozoon. This step may be executed with a conventional particle tracking algorithm. In this example, each region of interest ROI is a square with a side measurement of 50 pixels. FIG. 6A shows a detail of a detection image I_{d}, on which a region of interest ROIₚ, centred on a spermatozoon, is indicated. A particle tracking algorithm was used, for tracking each spermatozoon individually between different detection images. Each region of interest ROI is annotated with an identifier p denoting the spermatozoon. Each region of interest is denoted below as ROIₚ, where p is the annotation of the region of interest.

Step 130: forming a fluorescence emission image Iₘ of the sample, on the basis of the image I acquired by the image sensor. The fluorescence emission image Iₘ corresponds to a second spectral component, in the fluorescence detection band Δλm, of the image I acquired by the image sensor. On the basis of the annotated regions of interest ROIₚ of the detection image resulting from step 120, extracting what is called a twin region of interest, ROI′ₚ, from the fluorescence image. “Twin region of interest” is taken to mean a region of interest, extracted from the fluorescence image, which corresponds to the region of interest determined in the detection image in step 120. The region of interest ROIₚ, formed in the detection image I_{d}, and the twin region of interest ROI′ₚ, extracted from the fluorescence image Iₘ, correspond to the same particle p.

The detection and fluorescence images are obtained from the same image I acquired by the image sensor 20. Each region of interest ROIₚ defined in the detection image in step 120 is used to extract a twin region of interest ROI′ₚ from the fluorescence image. FIG. 6B shows a twin region of interest ROI′ₚ of the region of interest ROIₚ defined in FIG. 6A.

The advantage of using the same image sensor is that the detection image I_{d} and the fluorescence image Iₘ are formed from the same acquired image I. The regions of interest ROIₚ, ROI′ₚ, corresponding to the same particle p, are identical in each pair of images (detection image I_{d}, fluorescence image Iₘ).

When an image sensor having two elementary image sensors is used, as described with reference to FIG. 2B, the elementary image sensors are preferably identical and synchronized, so as to facilitate the correspondence between a region of interest ROIₚ defined in the detection image I_{d}, for a spermatozoon, and the twin region of interest ROI′ₚ, extracted from the fluorescence image Iₘ for the same spermatozoon.

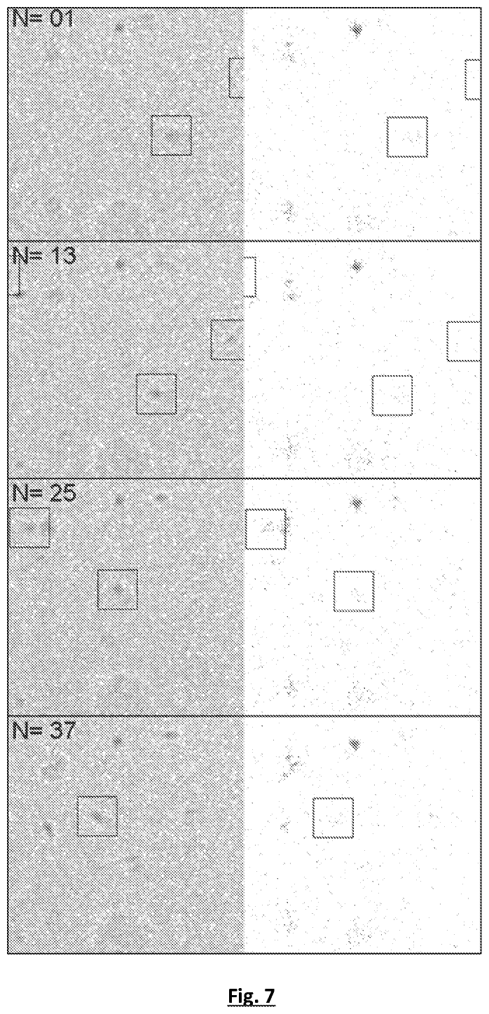

FIG. 7 shows various detection images I_{d} (left-hand column) and various fluorescence images Iₘ (right-hand column). In each detection image I_{d}, the trajectory of spermatozoa was tracked, and a region of interest was defined around each spermatozoon detected. The regions of interest defined on the basis of each detection image I_{d} were used for extracting twin regions of interest from each fluorescence image Iₘ: see the right-hand column. As described above, the regions of interest defined in each image are annotated so that each annotation corresponds to the same spermatozoon in the detection images and in the fluorescence images.

Each iteration of steps 100 to 130 may be assigned a rank n. n is a strictly positive integer. FIG. 7 shows the detection image and the fluorescence image formed on the basis of images acquired in the iterations of ranks 1, 13, 25 and 37.

Step 140: in this step, the twin regions of interest ROI′ₚ, having the same annotation, of each fluorescence image Iₘ resulting from step 130 are summed. This enables an integrated fluorescence image of each spermatozoon to be formed. “Integrated image” is taken to mean an image obtained by the integration of a plurality of images.

Thus,

$$\\ \begin{matrix}{I}_{p , N} = {\sum }_{1}^{N} {ROI}_{p , n}^{{}_{} ′} & ( 1 )\end{matrix}$$

where:
ROI′_{p,n} is a twin region of interest corresponding to the spermatozoon p defined in the fluorescence image acquired in the iteration n;
N corresponds to the total number of iterations;
I_{p,N} is the integrated fluorescence image after N iterations.

For each spermatozoon, as the number of cumulated fluorescence images increases, the signal-to-noise ratio also increases.

As a general rule, step 140 is a matter of forming an integrated fluorescence signal on the basis of regions of interest extracted from successive fluorescence images and corresponding to the same particle. The fluorescence signal may be the integrated fluorescence image I_{p,N}, corresponding to the cumulation of the region of interest extracted from a plurality of successive fluorescence images. It may also be a number corresponding to a cumulation of the intensities of the successive fluorescence images Iₘ, in the regions of interest extracted from each of them, and corresponding to the same particle.

According to one option, the regions of interest with the same annotation extracted from successively formed fluorescence images are averaged. Averaging enables the same dynamic to be retained for each fluorescence image.

Thus,

$$\\ \begin{matrix}{I}_{p , N} = \frac{{\sum }_{1}^{N} ROI {′}_{p , n}}{N} & ( 2 )\end{matrix}$$

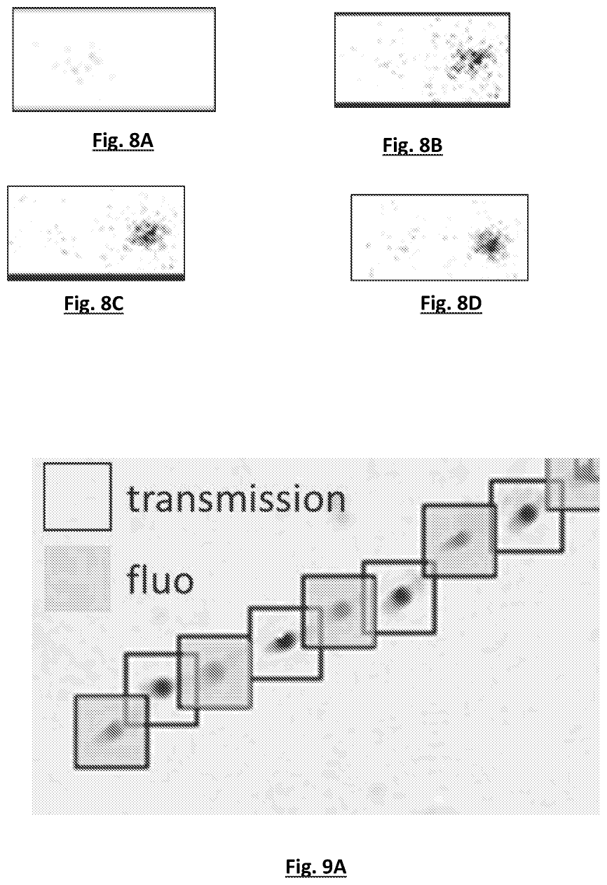

FIGS. 8A, 8B, 8C and show respectively, for the same spermatozoon p, the integrated fluorescence images I_{p,N}, as defined according to the corresponding equation (2), obtained, respectively after N=1 iteration (FIG. 8A), N=13 iterations (FIG. 8B), N=25 iterations (FIG. 8C) and N=47 iterations (FIG. 8D). The improvement of the signal-to-noise ratio as a function of the number of iterations, that is to say the number of averaged fluorescence images, can be observed visually.

Steps 100 to 140 are reiterated, the images of the sample being acquired at a high acquisition frequency, at 60 images per second for example. In FIG. 5A, it is assumed that the integrated fluorescence image I_{p,N} for each spermatozoon p is renewed in each iteration. Each iteration comprises steps 100 to 140. The iterations continue until a stop criterion is reached: there may be a predetermined number of iterations. The stop criterion may be defined according to the signal-to-noise ratio obtained in the integrated fluorescence image associated with each spermatozoon: above a predefined signal-to-noise ratio, the iterations cease for the spermatozoon whose associated integrated fluorescence image exceeds the signal-to-noise ratio threshold. The user may also decide to stop the iterations, on the basis of the visual rendering of each integrated fluorescence image.

According to another option, steps 100 and 110 are reiterated. Steps 120 to 140 are executed in post-processing, after an iteration of a predetermined number of iterations of steps 100 to 110. See FIG. 5B.

It is preferable for the detection images and the fluorescence images to be formed from the same image, or to be acquired simultaneously if a plurality of elementary sensors are used. However, it would be feasible for the respective image acquisitions for forming detection images and fluorescence images to be alternated. It is preferable to limit the time interval between the successive acquisitions of a detection image and a fluorescence image, in view of the movement of the spermatozoa. The period between two successive acquisitions may be such that the movement of the spermatozoa between a fluorescence image and a detection image is considered negligible. This facilitates the matching between the regions of interest ROIₚ determined in the detection images and the regions of interest determined in the fluorescence images. Alternatively, the position of the spermatozoa between two successive detection images may be estimated by interpolation, as described below.

The alternation of the detection images and the fluorescence images makes it possible to use a monochrome image sensor, which is more common in applications of microscopy.

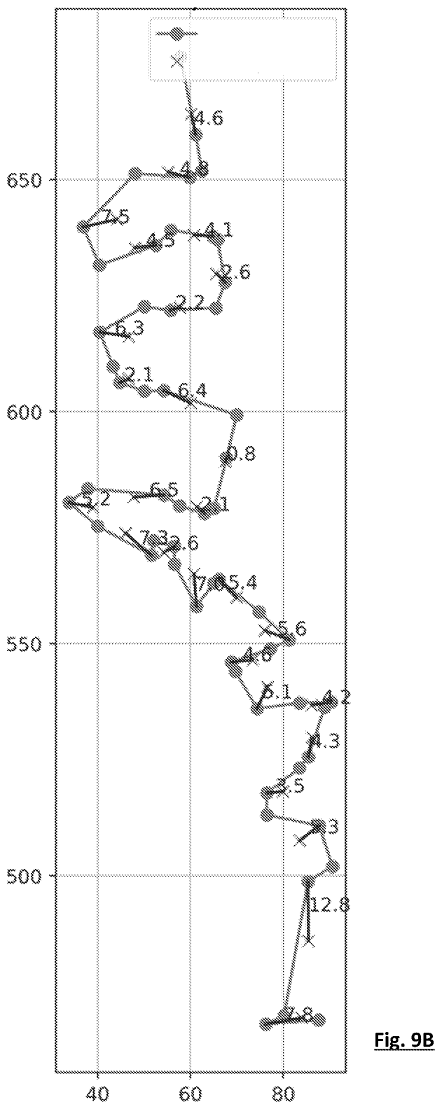

According to one option, illustrated in FIGS. 5C, 9A and 9B, the acquisitions of the detection images and the fluorescence images alternate. Thus steps 100 and 110 are temporarily time-shifted relative to each other during the same iteration. In this case, the method may comprise an interpolation step 125. The regions of interest ROI′_{p,n} extracted from a fluorescence image, in an iteration of rank n, are obtained by interpolation of regions of interest ROI_{p,n} and ROI_{p,n+1} corresponding respectively to the spermatozoon p and determined in the detection images of rank n and n+1.

FIG. 9A shows the regions of interest ROI_{p,n} and ROI_{p,n+1} detected in successive detection images of rank n and n+1, together with the interpolated region of interest ROI′_{p,n} assigned to the fluorescence image acquired between said detection images.

FIG. 9B shows a variation of the central point of regions of interest ROI_{p,n} detected in successive different detection images. Each point indicated by a circle, corresponds to a position of the central point. To test the interpolation algorithm, the position of central points of regions of interest ROI_{p,n+1} between two regions of interest ROI_{p,n}, ROI_{p,n+2} was estimated. The interpolated positions of the central points of the regions of interest ROI_{p,n+1} are indicated by crosses (X). The difference between the real position (circle) and the interpolated position (cross) is indicated by a linear segment. It may be observed that the interpolation becomes more correct as the trajectory of the spermatozoon becomes straighter.

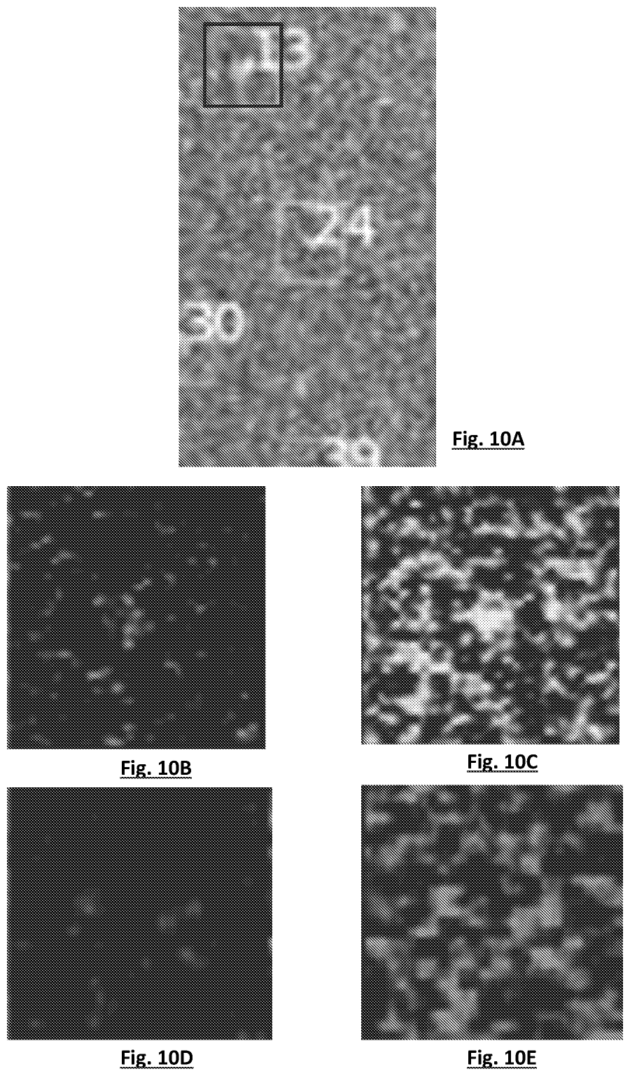

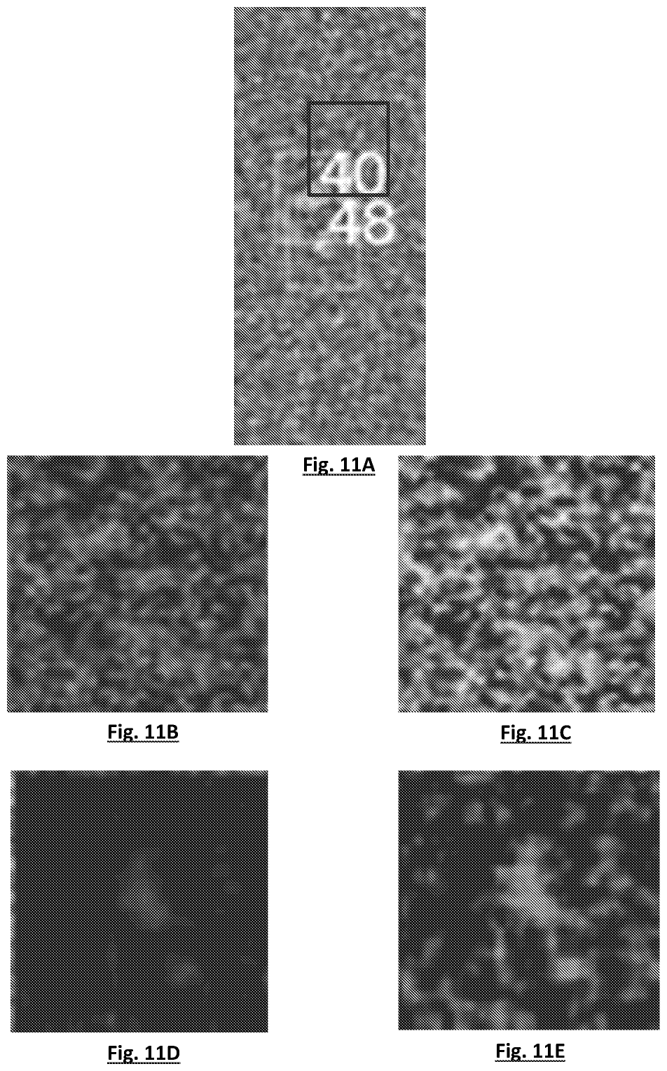

FIGS. 10A to 10E and 11A to 11E illustrate the option of applying the invention to two simultaneous measurements of fluorescence. To obtain these images, a device as shown schematically in FIG. 2A was used. The spermatozoa were marked with a fluorophore kit (EasyKit Viability, supplied by IMV), used for determining whether a spermatozoon is living or dead. The fluorophores used were SYBR green (Thermo Fisher) and propidium iodide (PI). Thus the living spermatozoa emitted a green fluorescence light and the dead spermatozoa emitted a red fluorescence light. The spectral excitation band was centred on 450 nm. The spectral detection band Δλd was centred on a wavelength of 430 nm (Thorlab FB430-10 filter).

FIGS. 10A and 11A correspond to two parts of a detection image I_{d}, extending around spermatozoa referenced 13 and 40 respectively. FIGS. 10B and 11B correspond, respectively, to the ROI′_{p.}, with p=13 and p=40, extracted from the image acquired by the image sensor in the green spectral band. FIGS. 10D and 11D correspond, respectively, to the ROI′_{p.}, with p=13 and p=40, extracted from the image acquired by the image sensor in the red spectral band. FIGS. 10C and 11C correspond, respectively, to the integrated fluorescence images based on N=10 annotated regions of interest ROI′_{p.}, with p=13 and p=40, extracted from the image acquired by the image sensor in the green spectral band. FIGS. 10E and 11E correspond, respectively, to the integrated fluorescence images based on N=10 annotated regions of interest ROI′_{p.}, with p=13 and p=40, extracted from the image acquired by the image sensor in the red spectral band.

The fluorescence images, considered in isolation from each other (see FIGS. 10B, 10D, 11B, 11D), do not allow conclusions to be drawn regarding the state of the spermatozoa referenced 13 and 40. From the integrated fluorescence images (see FIGS. 10C, 10E, 11C, 11E), it may be concluded that the spermatozoa referenced 13 and 40 are living (FIG. 10C) or dead (FIG. 11D), respectively.

Although it has been described in relation to spermatozoa, the invention is applicable to other particles, particularly biological particles. The particles may be cells, for example.

The invention is applicable to the detection of fluorescent particles, whether the fluorescence is exogenous, that is to say caused by marking with a fluorescent marker, or endogenous, as in the case of autofluorescence.

The invention may also be applied to the observation of luminescence, particularly bioluminescence, for example the bioluminescence of cells in a culture medium. In this case, the use of excitation is unnecessary, since bioluminescence is caused by a chemical reaction. Thus, in this embodiment, the device does not need to include an excitation source 12.

For both fluorescence and luminescence, the integration period, that is to say the period for which the regions of interest defined in each fluorescence image are cumulated, may vary from several seconds to several tens of minutes, or even longer. This is a further advantage of the invention: since the integration of fluorescence or luminescence images allows for the movement of the particles, it may be executed with a large number of images, over a long period.

1. A method for observing an emission of fluorescence light or luminescence light from a moving particle in a sample, the sample comprising particles moving in different directions, the particle emitting the fluorescence or luminescence light in a spectral emission band, the method comprising:
a) illuminating the sample in a spectral detection band, and forming a detection image of the sample in the spectral detection band, the spectral detection band being different from the spectral emission band;
b) forming an emission image of the sample in the spectral emission band;
the detection image and the emission image being obtained on the basis of an acquisition of an image of the sample by an image sensor, in the spectral detection band and in the spectral emission band,
wherein:
steps a) and b) are reiterated;
the method also comprises the following steps:
c) on the basis of each detection image resulting from step a) of each iteration of steps a) and b),
detecting the particle;
executing a tracking algorithm to detect the particle on each detection image;
determining a region of interest around the particle on each detection image;
d) on the basis of the region of interest resulting from each step c), extracting a region of interest from each emission image of the sample, the region of interest extracted from each emission image corresponding to the particle;
e) summing the regions of interest extracted in each step d) so as to form an integrated emission image of the particle, representative of the fluorescence or luminescence of the particle.

2. The method of claim 1, wherein:
the particle emits a fluorescence light in the spectral emission band when it is illuminated in a spectral excitation band;
step b) comprises illuminating the sample in the spectral excitation band;
the spectral detection band is remote from the spectral excitation band;
the image sensor is coupled to a filter so as to block the spectral excitation band.

3. The method of claim 1, wherein steps a) and b) are executed simultaneously.

4. The method of claim 3, wherein steps c), d) and e) are executed in each iteration of steps a) and b) or following the iterations of steps a) and b).

5. The method of claim 1, wherein, in each iteration, steps a) and b) are executed simultaneously.

6. The method of claim 5, wherein:
at least two iterations of steps a) and b) are executed successively, according to an iteration of rank n and an iteration of rank n+1, n being a strictly positive integer;
step d) comprises:
(i) interpolating the positions of the particle in the detection images resulting from steps a) of the iterations of rank n and n+1, so as to estimate an interpolated position of the particle in step b) of the iteration of rank n;
(ii) extracting the region of interest from the emission image formed in the iteration of rank n on the basis of the interpolated position estimated in sub-step (ii).

7. The method of claim 1, wherein:
the image sensor is a colour image sensor;
in step a), the detection image of the sample is obtained on the basis of a first spectral component of the image acquired by the image sensor, the first spectral component corresponding to all or part of the spectral detection band;
and/or, in step b), the emission image of the sample is obtained on the basis of a second spectral component of the image acquired by the image sensor, the second spectral component corresponding to all or part of the spectral emission band;
in such a way that the same image acquired by the image sensor may be used to form the detection image and the emission image of the sample.

8. The method of claim 1, wherein:
the image sensor comprises a first elementary image sensor and a second elementary image sensor, together with a beam splitter, the beam splitter being configured to send light,
in the spectral detection band, towards the first elementary image sensor;
and in the spectral emission band, towards the second elementary image sensor;
In each step a), the detection image, in the spectral detection band, is acquired by the first elementary image sensor;
in each step b), the emission image, in the spectral emission band, is acquired by the second elementary image sensor.

9. The method of claim 1, wherein step e) comprises calculating an average of the regions of interest, extracted from each emission image of the sample, corresponding to the particle.

10. The method of claim 1, wherein step e) is executed in each iteration of steps a) and b).

11. The method of claim 1, wherein:
the stop criterion is a predetermined number of steps;
or, step e) being executed in each iteration of steps a) and b), the stop criterion is the obtaining of an integrated emission image in which the signal-to-noise ratio exceeds a predetermined threshold,
or, step e) being executed in each iteration of steps a) and b), the method comprises a display of the integrated emission image, the iterations being stopped by a user.

12. The method of claim 1, wherein, in each step a), the intensity of illumination of the sample, in the spectral detection band, is adjusted so that the signal-to-noise ratio of the detection image is less than a predetermined value.

13. A device for observing a sample comprising a moving particle, the particle being capable of emitting a fluorescence light or a luminescence light in a spectral emission band, the device comprising:
a detection light source, configured to illuminate the sample in a spectral detection band, remote from the spectral emission band;
an image sensor, configured to acquire an image of the sample, in the spectral detection band and the spectral emission band;
the device being configured to keep the sample facing the image sensor on a sample plane;
an emission filter, placed between the image sensor and the sample plane, the emission filter being configured to transmit light in the spectral detection band and in the spectral emission band;
a processing unit configured to form, on the basis of the image acquired by the image sensor:
a detection image of the sample in the spectral detection band;
an emission image of the sample, in the spectral emission band;
wherein the processing unit is programmed to execute steps a) to e) of the method according to claim 1.

14. The device of claim 13, comprising an excitation light source configured to illuminate the sample in a spectral excitation band, remote from the spectral emission band and the spectral detection band.

15. The device of claim 14, wherein the detection light source and the excitation light source are configured to be activated simultaneously or sequentially, with a time shift of less than 100 ms or 10 ms.

16. The device of claim 13, wherein the image sensor is a colour image sensor.

17. The device of claim 13, wherein the image sensor comprises a first elementary image sensor and a second elementary image sensor, together with a beam splitter, the beam splitter being configured to send light,
in the spectral detection band, towards the first elementary image sensor;
and in the spectral emission band, towards the second elementary image sensor;
